(19) United States
(12) Patent Application Publication
(10) Pub. No.: US 2026/0221282 A1
(43) Pub. Date: Jul. 30, 2026

(54) TRAINING AND USE OF ARTIFICIAL INTELLIGENCE MODELS FOR PREDICTING THE OCCURRENCE OF COLORECTAL CARCINOMA OR ADVANCED COLORECTAL ADENOMA IN AN INDIVIDUAL

(71) Applicant: HUNA LTDA., São Paulo (BR)

(72) Inventors: Vinicius MOURA RIBEIRO, Laranjeiras (BR); Daniella CASTRO ARAUJO, Goiânia (BR); Marco Aurélio KOHARA, São Paulo (BR)

(21) Appl. No.: 19/331,910

(22) Filed: Sep. 17, 2025

Related U.S. Application Data
(63) Continuation-in-part of application No. 19/164,568, filed on Mar. 22, 2024.

(30) Foreign Application Priority Data
Mar. 23, 2023 (BR) 1020230053912

(51) Int. Cl. G16H 50/20

(52) U.S. Cl. CPC G16H 50/20

(57) ABSTRACT

The present invention relates to methods and systems for training an artificial intelligence model to predict the likelihood of a colorectal tumor or adenoma occurring in an individual, based on attributes obtainable from a routine blood test. The present invention also relates to methods and systems for predicting the likelihood of a colorectal tumor or adenoma occurring in an individual, based on the trained artificial intelligence model.

### CROSS-REFERENCE TO RELATED APPLICATIONS

This application is a continuation-in-part of U.S. patent application Ser. No. 19/164,568, filed Sep. 11, 2025, which is the National Stage in the United States of America of International Application No. PCT/BR2024/050106, filed Mar. 22, 2024. That application claims priority to Brazilian patent application No. 1020230053912, filed Mar. 23, 2023. The contents of all of those applications are incorporated by reference in their entireties.

### TECHNICAL FIELD

The invention relates to the technical field of artificial intelligence and, in particular, to methods and systems for assessing the risk of cancer occurring in an individual based on features obtained in a routine blood test.

### PRIOR ART

Colorectal cancer (CRC) is the second leading cause of cancer-related deaths worldwide, accounting for more than 900,000 deaths annually. Early detection of CRC is crucial for effective treatment and is responsible for reducing cancer-related mortality by 33-60%. Despite this, many patients do not adhere to recommended screening protocols, posing a major challenge to early diagnosis.

Currently, the most common methods for CRC screening are the fecal immunochemical test (FIT) and colonoscopy. FIT is a highly effective and widely available test that serves as a risk stratifier for colonoscopy. A recent study demonstrates that incorporating FIT into screening protocols significantly reduces the proportion of advanced-stage CRC diagnoses. However, adherence to FIT remains low due to psychological barriers, such as discomfort when handling samples of feces, and systemic factors, including low awareness and lack of recommendations from healthcare professionals.

These aforementioned limitations highlight the need for innovative approaches to support CRC screening efforts. Machine learning algorithms offer an affordable alternative for assessing cancer risk in large populations using personal health data.

Colonoscopy, while highly sensitive and effective in reducing CRC incidence and mortality, faces limitations in its widespread adoption. Basically, its use is limited by high costs, especially in resource-limited locations, together with procedural risks such as perforation and the discomfort of bowel preparation and anesthesia.

Recent studies demonstrate the potential of AI-enhanced complete blood count (CBC) tests in breast cancer and CRC screening. In this context, these studies propose the development of a machine learning model based on CBC tests as a risk stratification tool for CRC. By identifying high-risk individuals, this method can facilitate resource prioritization and active case finding in at-risk populations, complementing existing screening programs and improving overall accessibility.

### SUMMARY OF THE INVENTION

The present invention relates to a method for training an artificial intelligence model to predict the chances of colorectal carcinoma or advanced colorectal adenoma occurring in an individual, comprising at least the steps of: receiving a first set of features, wherein the first set of features is related to individuals who have not been diagnosed with colorectal carcinoma or advanced colorectal adenoma and comprises, as features for each individual, at least one feature obtained from the red series of a blood count and/or at least one feature obtained from the white series of a blood count; receiving a second set of features, wherein the second set of features is related to individuals diagnosed with colorectal carcinoma or advanced colorectal adenoma and comprises, as features for each individual, at least one feature obtained from the red series of a blood count and/or at least one feature obtained from the white series of a blood count; and training the artificial intelligence model based on the first and second sets of features.

Optionally, the first and second sets of features additionally comprise at least one feature obtained from the platelet series of a blood count.

Optionally, the first and second sets of features additionally comprise age as a feature for each individual.

Optionally, the model is trained using a supervised or unsupervised learning algorithm, or comprises a combination of models trained using a supervised and/or unsupervised learning algorithm.

The present invention also relates to a system for training an artificial intelligence model to predict the chances of colorectal carcinoma or advanced colorectal adenoma occurring in an individual, the system comprising at least one processor, wherein the processor is configured to carry out the method for training an artificial intelligence model as described above.

The present invention also relates to a computer-readable medium comprising instructions that, when executed by at least one processor, cause the processor to carry out the method for training an artificial intelligence model as described above.

The present invention also relates to a method for predicting the chances of colorectal carcinoma or advanced colorectal adenoma occurring in an individual, comprising the steps of: receiving, as features for the individual, at least one feature obtained from the red series of a blood count and/or at least one feature obtained from the white series of a blood count; and calculating, using a model trained according to the method for training an artificial intelligence model as described above and based on the received features, the chances of colorectal carcinoma or advanced colorectal adenoma occurring in the individual.

Optionally, the first and second sets of features additionally comprise age as a feature for each individual.

Optionally, the first and second sets of features additionally comprise at least one feature obtained from the platelet series of a blood count.

The present invention also relates to a system for predicting the chances of colorectal carcinoma or advanced colorectal adenoma occurring in an individual, the system comprising at least one processor, wherein the processor is configured to carry out the method for predicting the chances of colorectal carcinoma or advanced colorectal adenoma occurring in an individual as described above.

The present invention also relates to a computer-readable medium comprising instructions that, when executed by at least one processor, cause the processor to carry out the method for predicting the chances of colorectal carcinoma or advanced colorectal adenoma occurring in an individual as described above.

### BRIEF DESCRIPTION OF THE DRAWINGS

FIG. 1 illustrates an exemplary architecture of a system according to the present invention.

FIG. 2 illustrates a flowchart of a study population according to an exemplary embodiment of the present invention.

FIG. 3a illustrates a performance graph of a Ridge regression model for colorectal cancer (CRC) and advanced adenoma according to the exemplary embodiment of the present invention.

FIG. 3b illustrates a performance graph of a Ridge regression model for colorectal cancer (CRC) stratified by sex according to the exemplary embodiment of the present invention.

FIG. 3c illustrates a performance graph of a Ridge regression model for colorectal cancer stratified by sex according to the exemplary embodiment of the present invention.

FIG. 4 illustrates a graph of Ridge regression coefficients illustrating the contributions of each feature to the model according to the exemplary embodiment of the present invention.

FIG. 5 illustrates a performance graph of the Ridge regression model according to tumor invasion status according to the exemplary embodiment of the present invention.

FIG. 6 illustrates a cumulative gain graph of the risk distribution for colorectal cancer according to the exemplary embodiment of the present invention.

### DETAILED DESCRIPTION

The present invention relates to artificial intelligence techniques applied in the prediction of the occurrence of cancer in an individual. The inventors discovered that, based on features obtainable from routine blood tests (CBC), it is possible to create and apply models with an adequate accuracy rate in predicting the occurrence of colorectal carcinoma or advanced colorectal adenoma.

A CBC is commonly divided into three groups: white series (leukogram); red series (erythrogram); and platelet series (platelet count). The red series evaluates the parameters related to red blood cells. The white series evaluates the different types of white blood cells. The platelet series is primarily intended for counting and analyzing platelets.

Within the context of the present invention, a feature obtainable from a routine blood test is one that comprises one or more values obtained from the red series, the white series, and/or the platelet series. The red series is the group that includes, for example, values for: hemoglobin, red blood cell count (RBC), hematocrit, mean corpuscular volume (MCV), mean corpuscular hemoglobin (MCH), mean corpuscular hemoglobin concentration (MCHC), and red blood cell distribution width (RDW). The white series is the group that includes, for example, values for: leukocytes, neutrophils, rods, basophils, eosinophils, lymphocytes, monocytes, and blasts. The platelet series is the group that includes, for example, values for: platelets and mean platelet volume (MPV).

Naturally, features can be obtained from the combination of features (e.g., ratio or any formula that combines features) within the same series and from features between different series. Examples include: aggregated systemic inflammation index (neutrophils×monocytes×(platelets/lymphocytes)) (AISI), derived neutrophil-to-leukocyte ratio (neutrophils/(lymphocytes-neutrophils)) (dNLR), lymphocyte-to-monocyte ratio (LMR), neutrophil-to-lymphocyte ratio (NLR), platelet-to-lymphocyte ratio (PLR), systemic immune inflammation index (platelets×(neutrophils/lymphocytes)) (SII), systemic inflammation response index (neutrophils×(monocytes/lymphocytes)) (SIRI), hemoglobin-to-platelet ratio (HPR).

In the context of the present invention, the set of features obtainable in routine blood tests may also include an individual's age, which is usually provided by the individual when performing such tests or may be obtained directly from the individual or from some record of that individual, and any relationship between age and any other feature. All of these features are easily obtained, not requiring the use of any complex or costly techniques or equipment.

It should be understood that, in addition to these features above, other features, even if not obtainable through routine blood tests, can be added for training and use of the models. Examples include features obtained from imaging tests, features related to an individual's phenotypic characteristics, and features obtained from routine medical examinations, such as in-office exams. Examples include the individual's family history, obesity, sedentary lifestyle, smoking, alcohol consumption, and chronic inflammatory bowel disease.

The model training method according to the present invention may optionally include a prior step of collecting blood from one or more individuals to obtain the above features.

A model can be trained generically from two sets of features: features obtained from individuals diagnosed with colorectal carcinoma or advanced colorectal adenoma, or who have developed colorectal carcinoma or advanced colorectal adenoma after a certain period of time from the blood collection; features obtained from individuals who have not been diagnosed with colorectal carcinoma or advanced colorectal adenoma and who have not developed colorectal carcinoma or advanced colorectal adenoma after a certain period of time from the blood collection.

For training a model, known machine learning techniques can be employed, such as supervised and unsupervised learning techniques. Examples of supervised learning techniques include decision trees, k-Nearest Neighbors algorithms, Naïve Bayes, and different types of regression. Examples of unsupervised learning techniques include genetic algorithms, neural networks, Support Vector Machines, and regression and fuzzy algorithms.

In the context of the present invention, a model can also comprise a combination of models trained using different techniques. This combination is known in the prior art as an ensemble.

Once trained, the model acts as a classifier, receiving features as input and outputting the predicted occurrence of colorectal carcinoma or advanced colorectal adenoma in an individual.

For training a model and for using a trained model, the features can be encoded or not. In the absence of coding, features as measured and reported according to standards applied in routine blood tests can be used. An example of coding is the application of some normalization technique to input features.

In a preferred embodiment of the present invention, at least one feature obtained from the red series of a blood count and/or at least one feature obtained from the white series of a blood count are used as features for training and using a model.

In another preferred embodiment of the present invention, an individual's age, at least one feature obtained from the red series of a blood count, and at least one feature obtained from the white series of a blood count are used as features for training and using a model.

In another preferred embodiment of the present invention, an individual's age, at least one feature obtained from the red series of a blood count, at least one feature obtained from the white series of a blood count, and at least one feature obtained from the platelet series of a blood count are used as features for training and using a model.

A computer-readable medium is any medium capable of being accessed by a processor and capable of storing instructions that can be read by the processor for the execution of a method. Examples include flash drives, external and internal hard drives, CD-ROMs, and RAM and ROM memories.

FIG. 1 illustrates an example of a client/server system 1 that can be implemented for the purposes of the present invention. As can be seen, one or more client devices 10-n are connected to a server and communicate through an interface 30.

The client devices 10-n include one or more processors 11, a memory set 12, and data input and output interfaces 13, and can be implemented as personal devices or server terminals, taking the form, for example, of laptops, desktops, tablets, or smartphones.

The data input and output interfaces 13 may be, for example, a keyboard, a mouse, a microphone, a speaker and a touch screen.

Interface 30 may be a wired or wireless communication interface, such as a WAN, LAN, or the Internet.

The server includes one or more processors 21 and a memory set 22 and may be implemented as a personal computer, a network server, or a virtual server.

Memory sets 12 and 22 may include transient and non-transitory computer-readable media, such as ROM and RAM. Memory sets 12 and 22 typically store both the operating systems of the client and server devices and computer-readable instructions for training and using models based on the features of one or more individuals.

In example system 1, a model may be trained by the one or more processors 21 of the server and also stored in the memory set 22 of the server. Alternatively, the model can be trained on another processing device, such as a client device, and stored in the server's memory set 22. Alternatively, the model can be trained by the server and one or more client devices jointly.

With a trained model, a client device 1-n can, through the data input and output interfaces 13, receive and send features associated with an individual to the server. Upon receiving the features, the server's processor 21 uses them as input for the trained model, which estimates the chances of colorectal cancer occurring in the individual associated with those features. These chances of occurrence are then made available to the client device 1-n. Alternatively, a client device 1-n can receive and send features of more than one individual to the server. In the same manner, the server receives and processes such features, returning the chances of colorectal carcinoma or advanced colorectal adenoma occurring in all individuals associated with such features.

As anyone skilled in the art will appreciate, system 1 in FIG. 1 does not need to be implemented in a client/server architecture. In this situation, there is no server, the processor and memory set of a client device, such as a personal computer, being responsible for training and/or storing the model and executing the instructions for calculating the chances of cancer occurring in an individual.

The method for predicting the chances of cancer occurring in an individual based on the models trained according to the present invention may optionally comprise a prior step of collecting blood from one or more individuals to obtain the features.

Additionally, based on the chances of cancer occurring in an individual, a physician may establish treatment procedures for that individual and/or procedures for performing additional medical examinations on that individual.

### EXAMPLES OF EMBODIMENTS

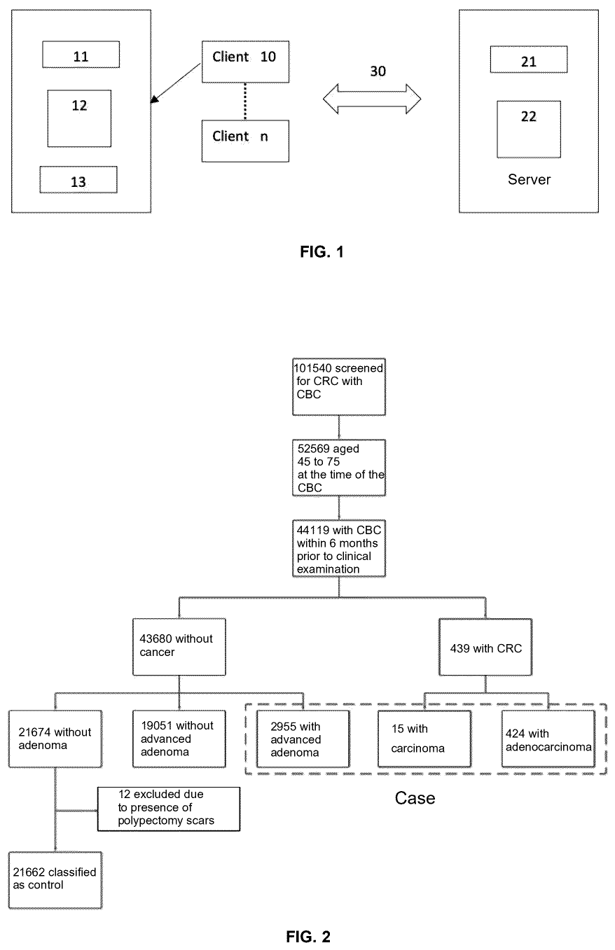

FIG. 2 shows a flowchart of a study population from the Fleury laboratory according to an example embodiment of the present invention.

For training the models used in the examples of embodiments of the present invention below, two supervised machine learning techniques were employed. Two models were trained using ridge regression, as described in “Hoerl, A. E. & Kennard, R. W. Ridge regression: Biased estimation for nonorthogonal problems. Technometrics 12, 55 (1970).” Two models were trained using the Gradient Boosting (GBM) technique, using the LightGBM algorithm described in “Guyon I, Von Luxburg U, Bengio S, Wallach H, Fergus R, Vishwanathan S, et al. LightGBM: A Highly Efficient Gradient Boosting Decision Tree. Von Luxburg and S. Bengio and H. Wallach and R. Fergus and S. Vishwanathan and R. Garnett IGAU, editor. Curran Associates, Inc.; 2017”.

The performance of each generated model was evaluated using the AUC (area under the curve) measure as described in “Fawcett T. an introduction to ROC analysis. Pattern Recognition Letters. 2006. pp. 861-874. doi: 10.1016/j.patrec.2005.10.010”. The AUC performance was obtained by plotting the true positive rate as a function of the false positive rate at varying thresholds. Since the model output is a probability, i.e., the risk of cancer occurrence, the threshold ranges from 0 to 1. In each performance evaluation, a 95% confidence interval (CI) is predicted, ensuring statistical significance. This was achieved by performing 1,000 bootstrap resamplings with repositioning of the training set.

In addition to age, the following features obtainable from a blood test were tested: eosinophils; hematocrit; hemoglobin; leukocytes; lymphocytes; MCH; MCHC; MCV; monocytes; neutrophils; platelets; RBC and RDW. The following derived features were also tested: NLR; AISI; dNLR; LMR; PLR; HPR; SII and SIRI. In total, 22 features were tested.

From the set of 22 features listed above, a directed acyclic graph method was used to identify the most relevant ones. Starting with single-feature models, the one with the highest AUC was selected. At each step, an additional feature was added, and the best-performing model (by AUC) was retained. This iterative process continued until the AUC gain fell below its standard deviation, balancing performance with model simplicity. Features were normalized using the minimum and maximum values from the training set to maintain a consistent scale.

Since the dataset had no missing values, no imputation was necessary. Due to the higher incidence of CRC in men than in women and its low prevalence in the general population, the target variable was significantly imbalanced in the training and validation sets (2.04% and 1.98%, respectively). To mitigate this, higher weights were assigned to CRC cases during model training, with even higher weights applied to female cases to account for their lower incidence.

To evaluate classification performance, the Ridge regression model was compared with the Advanced Tabular Probabilistic Network (TabPFN). TabPFN is a transformer-based classifier trained via meta-learning, capable of producing accurate predictions. Final predictions were obtained by aggregating results from all members of the ensemble.

After applying the best-performing model, a classification framework described by “Araujo, D. C. et al. Unlocking the complete blood count as a risk stratification tool for breast cancer using machine learning: a large-scale retrospective study. Sci. Rep. 14, 10841 (2024)” was used to assign individuals to three distinct risk groups-high, moderate, and medium-based on the predicted probabilities generated by the model. For each group, the relative risk (RR) was calculated, defined as the proportion of cases within the group divided by its share of the average population.

### Example—Prediction of Colorectal Carcinoma Occurrence

In this example embodiment, the study included 44, 119 individuals, of which 43,680 (99%) had no cancer diagnosis, and 439 were diagnosed with CRC, as shown in FIG. 2. In the cancer-free group, 21,674 had no adenomas, 19,051 had non-advanced adenomas, and 2,955 had advanced adenomas, classified as described in “Edge, S. B. & Compton, C. C. The American Joint Committee on Cancer: the 7th edition of the AJCC cancer staging manual and the future of TNM. Ann. Surg. Oncol. 17, 1471-1474 (2010)”. An adenoma was considered advanced if it met any of the following criteria: diameter≥10 mm, villous architecture≥25%, high-grade dysplasia, or early invasive cancer.

For model training and evaluation, 19,063 individuals with non-advanced adenomas or polypectomy scars were excluded. The final dataset included 25,056 individuals: 439 (1.8%) with CRC, 2,955 (11.8%) with advanced adenomas (defined as cases), and 21,662 (86.5%) classified as controls. The dataset was divided into training (70%) and validation (30%) sets. The training set included 17,538 individuals (9,430 women, 53.77%; 8,108 men, 46.23%), comprising 310 (2.04%) CRC cases, 2,066 (13.63%) advanced adenoma cases, and 15,162 (84.33%) controls. The validation set included 7,517 individuals (4,063 women, 54.05%; 3,454 cases, 45.95%), comprising 129 (1.98%) CRC cases, 889 (13.68%) advanced adenoma cases, and 6,499 (84.34%) controls.

Carcinoma subtypes and tumor staging were extracted from pathology reports. Among 439 CRC cases, 84 (19.1%) had no information available. For the remaining 355, 86 (24.3%) were classified as in situ and 268 (75.7%) as invasive. Of the 268 invasive cases, tumor staging was extracted from 47 (17.5%) using the American Joint Committee on Cancer (AJCC) TNM system for colorectal cancer.

In this example embodiment, complete blood counts (CBC) were analyzed. 25,056 individuals were included, of whom 439 (1.8%) were diagnosed with colorectal cancer (CRC), 2,955 (11.8%) had advanced adenomas, and 21,662 (86.5%) were classified as controls.

Descriptive analysis revealed significant differences between CRC cases and controls in most CBC markers, CBC-derived ratios, and age (P<0.001), with the exception of lymphocytes (Table 1). RDW, platelets, leukocytes, monocytes, neutrophils, and eosinophils levels were elevated in CRC cases, while red blood cell (RBC), hematocrit, hemoglobin, MCH, MCHC, and MCV levels were significantly lower compared to controls. Furthermore, six of the eight CRC-derived ratios were elevated in CRC cases, while LMR and HPR were reduced.

When comparing advanced adenomas with controls, significant differences were also observed in most blood count markers, CBC-derived ratios, and age, with the exception of lymphocytes, eosinophils, MCHC, and PLR.

Statistical significance was determined using Student's t-test, with P values≤0.002 considered significant. P values were adjusted for multiple testing using the Bonferroni correction method (the alpha value was corrected from 0.05 to 0.002).

Predictive AUCs were estimated by training a two-level decision tree on the training set and evaluating it on the validation set. To calculate the 95% confidence interval, bootstrap resampling was performed 1,000 times on the training set.

TABLE 1
Descriptive statistics of blood count (CBC) markers and
age among colorectal cancer (CRC) cases and controls.

| | | | | | Predictive AUC |
|---|---|---|---|---|---|
| Exam | Variable | Controls | CRC | P-value | (CI 95%) |
| | Age (years) | 53.64 ± 6.84 | 59.15 ± 8.05 | 0.000 | 0.60 (0.60-0.60) |
| CBC | Eosinophils (/mm³) | 195.02 ± 178.45 | 222.35 ± 201.31 | 0.002 | 0.52 (0.50-0.53) |
| | Hematocrit (%) | 42.01 ± 3.54 | 40.52 ± 5.22 | 0.000 | 0.52 (0.51-0.53) |
| | Hemoglobin (g/dL) | 14.11 ± 1.36 | 13.37 ± 2.03 | 0.000 | 0.52 (0.51-0.52) |
| | Leukocytes (/mm³) | 6166.83 ± 1881.98 | 6910.80 ± 2419.67 | 0.000 | 0.54 (0.52-0.54) |
| | Lymphocytes (/mm³) | 1975.79 ± 1014.36 | 1965.79 ± 1097.26 | 0.838 | 0.51 (0.50-0.52) |
| | MCH (pg) | 30.00 ± 1.98 | 29.02 ± 3.09 | 0.000 | 0.52 (0.51-0.52) |
| | MCHC (%) | 0.003358 ± 0.000104 | 0.003291 ± 0.000153 | 0.000 | 0.52 (0.51-0.53) |
| | MCV (fL) | 0.89 ± 0.05 | 0.88 ± 0.07 | 0.000 | 0.52 (0.51-0.53) |
| | Monocytes (/mm³) | 501.78 ± 172.18 | 589.23 ± 245.21 | 0.000 | 0.54 (0.53-0.55) |
| | Neutrophils (/mm³) | 3457.33 ± 1302.31 | 4093.53 ± 1723.79 | 0.000 | 0.54 (0.53-0.54) |
| | Platelets (10³/mm³) | 239.39 ± 59.89 | 257.04 ± 85.76 | 0.000 | 0.52 (0.50-0.52) |
| | RBC (10⁶/mm³) | 4.71 ± 0.45 | 4.62 ± 0.58 | 0.000 | 0.51 (0.50-0.52) |
| | RDW (%) | 13.33 ± 1.03 | 14.11 ± 1.87 | 0.000 | 0.53 (0.50-0.54) |
| CBC rates | AISI (×10⁶) | 239.18 ± 229.23 | 418.49 ± 565.49 | 0.000 | 0.53 (0.52-0.54) |
| | dNLR | 1.34 ± 0.56 | 1.57 ± 0.95 | 0.000 | 0.53 (0.52-0.53) |
| | HPR | 0.000063 ± 0.000019 | 0.000059 ± 0.000024 | 0.000 | 0.51 (0.50-0.53) |
| | LMR | 4.22 ± 1.79 | 3.63 ± 1.62 | 0.000 | 0.54 (0.53-0.55) |
| | NLR | 1.89 ± 0.98 | 2.41 ± 2.12 | 0.000 | 0.53 (0.53-0.53) |
| | PLR | 131.24 ± 49.32 | 149.51 ± 89.05 | 0.000 | 0.52 (0.51-0.53) |
| | SII (×10³) | 454.28 ± 279.01 | 648.27 ± 692.14 | 0.000 | 0.52 (0.51-0.53) |
| | SIRI | 980.27 ± 762.90 | 1474.80 ± 1522.71 | 0.000 | 0.55 (0.53-0.55) |

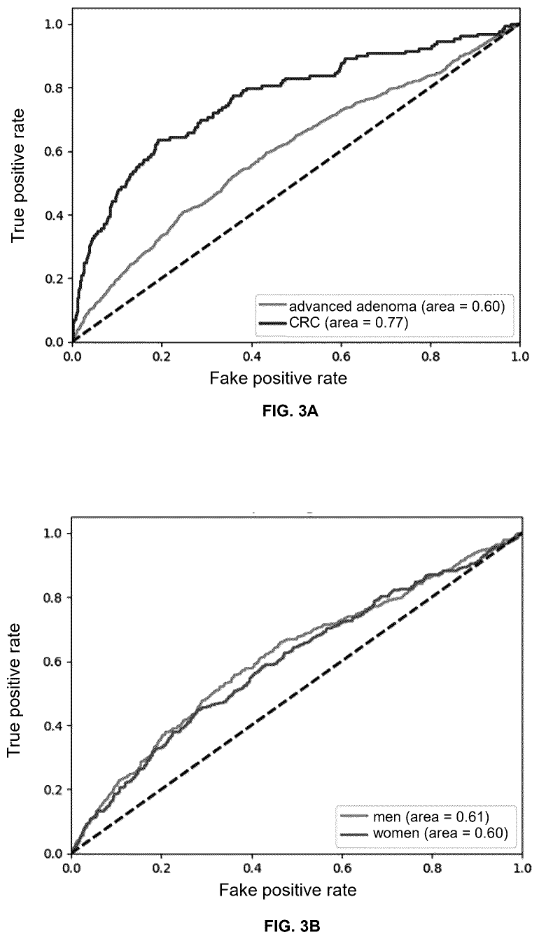

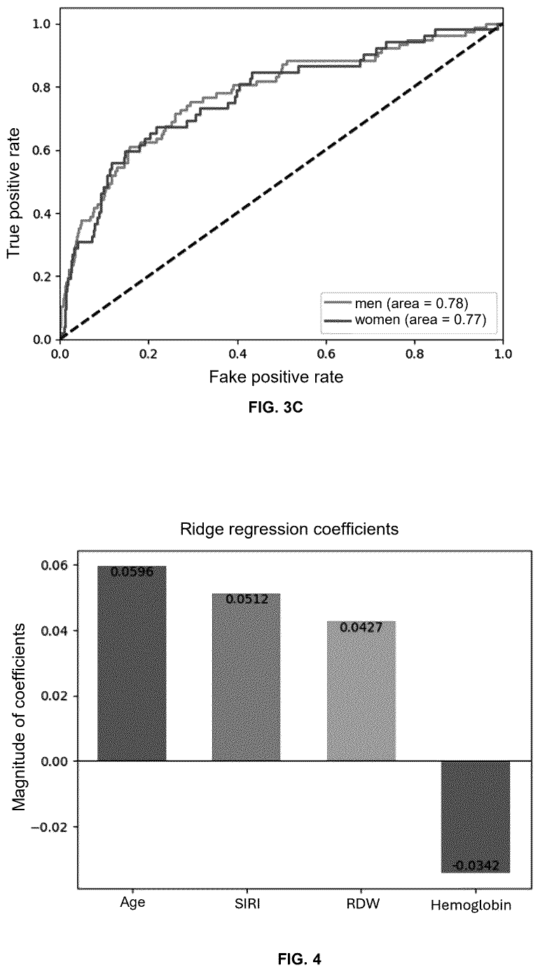

Using a variable selection methodology, the markers RDW, SIRI, hemoglobin, and age were incorporated into a Ridge regression model, achieving an AUC of 0.77 (95% CI: 0.75-0.77) for colorectal cancer (CRC) and 0.60 (95% CI: 0.58-0.61) for advanced adenomas (FIG. 3a).

For comparison purposes, the Ridge and TabPFN models were trained using the same set of variables. TabPFN achieved an AUC of 0.77 (95% CI: 0.73-0.81) for CRC and 0.63 (95% CI: 0.61-0.65) for advanced adenomas. Although TabPFN performed slightly better for advanced adenomas compared to Ridge regression, it showed greater variability and lower stability. Also considering the better interpretability of the Ridge model, it was selected for subsequent analyses.

To assess potential gender-related biases, the model was trained with the combined dataset and then evaluated separately in subsets of women and men. For CRC, the AUC was 0.77 (95% CI: 0.76-0.78) in women and 0.78 (95% CI: 0.77-0.78) in men (FIG. 3b). For advanced adenomas, the AUC was 0.60 (95% CI: 0.59-0.61) in women and 0.61 (95% CI: 0.60-0.62) in men (FIG. 3c). Although the differences were small, they were statistically significant (P<0.001).

### Example—Ridge Regression Model Explainability

The influence of individual variables on the decision-making process was analyzed by evaluating the coefficients of the Ridge regression model. The explainability analysis revealed that older age, higher RDW and SIRI levels, and lower hemoglobin levels were associated with a higher risk of colorectal cancer (CRC) (FIG. 4).

### Evaluating Ridge Model Performance According to Tumor Invasion Grade

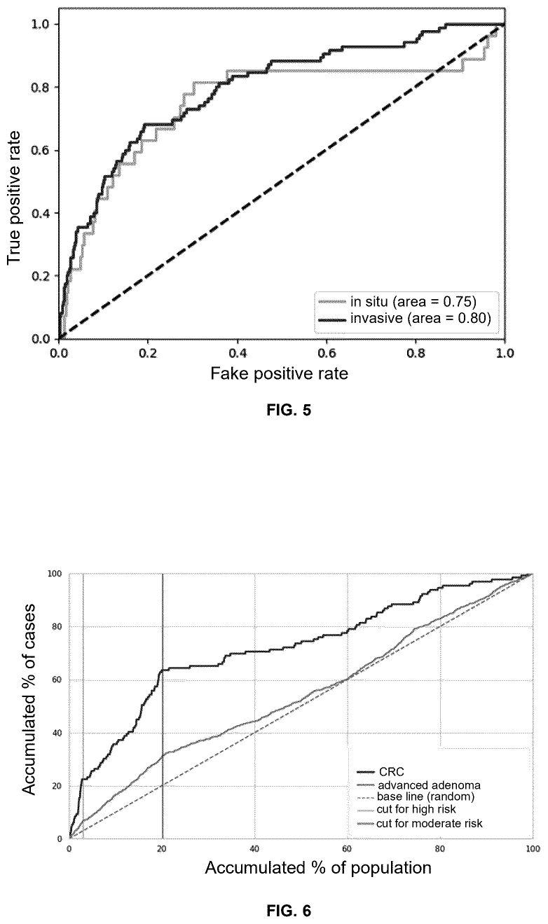

The Ridge model's performance was evaluated at different stages of colorectal cancer (CRC) tumor invasion by measuring the AUC for specific subsets of the validation case group. Each subset included cases with a specific degree of invasion, along with all samples from the control group. The model performed better for invasive tumors (AUC=0.80) compared to in situ tumors (AUC=0.75) (FIG. 5).

### Example—Comparative Analysis Between FIT and the Complete Blood Count (CBC)-Based Model

Of a total of 25,056 individuals, 1,817 (7.25%) had FIT test results obtained within a six-month period centered on the baseline clinical examination. This subset consisted of 981 women (54%) and 836 men (46%). Among these individuals, 58 were diagnosed with colorectal cancer (CRC), of which 88% tested positive on the FIT. Among the 1,499 individuals in the control group, 23% tested positive on the FIT (Table 2).

TABLE 2
Distribution of cases (CRC and advanced adenomas)
and controls according to the FIT test result.

| | | FIT (−) | FIT (+) | Total |
|---|---|---|---|---|
| Controls | | 1,160 | 339 | 1,499 |
| Cases | CRC | 7 | 51 | 58 |
| | AA | 108 | 152 | 260 |

| | | | |
|---|---|---|---|
| Total | 1,275 | 542 | 1,817 |

The sensitivity and specificity of FIT were compared with the complete blood count (CBC)-based model, using a threshold that maximized balanced accuracy. For colorectal cancer (CRC), FIT achieved 88% sensitivity and 77% specificity, while the model of the present invention achieved 64% sensitivity and 81% specificity. For advanced adenomas, FIT achieved 58% sensitivity and 77% specificity, compared with 32% sensitivity and 81% specificity of the CBC model of the present invention (Table 3).

TABLE 3
Comparison of metrics between the FIT test and the CBC-based
model for colorectal cancer (CRC) and advanced adenoma.

| | | | | |
|---|---|---|---|---|
| Sensitivity | Specificity | Accuracy | PPV | NPV |

CRC

| | | | | | |
|---|---|---|---|---|---|
| FIT | 88% | 77% | 78% | 13% | 99% |
| CBC | 64% | 81% | 80% | 6% | 99% |

Advanced Adenoma

| | | | | | |
|---|---|---|---|---|---|
| FIT | 58% | 77% | 75% | 31% | 91% |
| CBC | 32% | 81% | 75% | 18% | 90% |

### Example—Risk Stratification

To define the average risk group for colorectal cancer (CRC), the average annual incidence of CRC in Brazil—approximately 0.03%—was first estimated based on public data from the National Health Agency (ANS), accessed at: “Open Data Portal. https://dados.gov.br/dados/organizacoes/visualizar/agencia-nacional-de-saude-suplementar.” Individuals were then ranked in ascending order of predicted probability, and those with the lowest scores were cumulatively included until the proportion of CRC cases in this group equaled the national incidence. The 3% with the highest predicted probabilities were classified as high risk, while individuals in the intermediate range were assigned to the moderate risk group.

This risk stratification approach yielded relative risk (RR) values of 16.48, 5.22, and 1.0 for the high, moderate, and average risk groups, respectively. The high-risk group comprised only 3% of the population but accounted for 22.5% of all CRC cases. The moderate-risk group included 17% of individuals and accounted for 41.1% of CRC cases, while the average-risk group, representing the remaining 80% of the population, accounted for 36.4% of CRC cases.

When the same thresholds were applied to advanced adenomas, 6.9% of cases were identified in the high-risk group (RR=2.57), 25.4% in the moderate-risk group (RR=1.66), and 67.7% in the average-risk group (RR=1.0).

As shown in FIG. 6, the cumulative gain curve highlights the model's ability to concentrate CRC cases in a small fraction of the population. Specifically, the top 3% (high-risk cutoff, represented by the orange line) and the top 20% (moderate-risk cutoff, represented by the red line) of individuals, corresponding to the inflection points of the curve, captured 22.5% and 64% of CRC cases, respectively-substantially outperforming random allocation. For advanced adenomas, the top 3% and 20% of individuals represented 6.9% and 35% of cases, respectively.

Therefore, as demonstrated in this embodiment example, the present invention proposes a technical solution where markers obtained from complete blood counts (CBC) can help identify individuals at high risk of colorectal cancer (CRC) up to six months before diagnosis. Because CBCs are commonly included in routine medical examinations, this implementation utilizes existing healthcare infrastructure without substantially increasing costs.

Descriptive analysis showed significant differences between CRC cases and control cases for most CBC markers, CBC-derived ratios, and age (P<0.001), with the exception of lymphocytes, suggesting their potential as predictive markers for CRC.

Artificial intelligence was used to develop a Ridge regression model incorporating age, SIRI, RDW, and hemoglobin. This model achieved an AUC of 0.77 (95% CI: 0.75-0.77) for CRC and 0.60 (95% CI: 0.58-0.61) for advanced adenomas. Its performance was comparable to that of a more complex deep learning model, TabPFN, offering greater stability, interpretability, and lower computational complexity.

Explanatory analysis of this preferred embodiment indicates that older age, increased SIRI and RDW, and lower hemoglobin levels are associated with an increased risk of CRC. Age is correlated with cancer, including CRC, due to the cumulative effect of exposure to environmental carcinogens and DNA replication errors.

Systemic inflammation is associated with shorter survival in CRC patients. Inflammation is estimated to contribute to tumor initiation, progression, invasion, and metastasis in up to 50% of cancer cases. SIRI, an inflammatory index calculated from neutrophil, monocyte, and lymphocyte counts, has been identified as a prognostic marker in cancer. In CRC patients, elevated SIRI levels correlate with worse survival outcomes.

An elevated SIRI, driven by high neutrophil and monocyte counts and/or low lymphocyte levels, suggests a greater potential for tumor spread or recurrence, in addition to a weakened immune response. In this example, elevated monocyte and neutrophil levels were observed in CRC patients. However, there was no significant difference in lymphocyte counts compared to the control group.

Neutrophils promote cancer progression by inducing angiogenesis, immunosuppression, and metastasis, in addition to creating an inflammatory environment that favors tumor growth. Monocytes contribute to tumor-associated inflammation and are linked to the density of tumor-associated macrophages, creating a microenvironment favorable to the development of CRC. In contrast, higher lymphocyte counts are associated with a higher survival rate of individuals with CRC.

Similarly, an elevated RDW (erythrocyte anisocytosis index) is associated with a worse prognosis in patients with CRC. RDW measures the variation in red blood cell size. An elevated RDW usually precedes other changes in the blood count, such as low mean corpuscular volume (MCV) or hemoglobin, and is an early indicator of iron deficiency.

Furthermore, low hemoglobin levels are linked to anemia, inflammation, and increased tumor aggressiveness. In patients with CRC, the prevalence of anemia ranges from 30% to 75%. The causes of anemia in cancer patients are primarily attributed to systemic inflammation and iron deficiency, which can result from gastrointestinal bleeding associated with tumor ulceration.

Among the 439 CRC cases, tumor invasion status could be determined in 355. Of these, 24.3% were classified as in situ and 75.7% as invasive. However, detailed classification using the TNM system was available for only 17.5% of invasive cases, which limited the ability to conduct a more accurate analysis of CRC progression.

When evaluating the Ridge model's performance for in situ versus invasive carcinomas, the model performed better for invasive tumors (AUC=0.80) compared to in situ (AUC=0.75), suggesting more distinct biological patterns as the carcinoma progresses. Consistently, previous studies have shown that changes in blood count parameters, such as hemoglobin levels and white blood cell counts, are more pronounced in advanced-stage CRC than in in situ tumors.

Fecal immunoassay testing (FIT) is the primary noninvasive method for CRC screening and has been shown to significantly reduce mortality from the disease. In this embodiment, FIT demonstrated robust predictive performance for detecting CRC (sensitivity: 88%, specificity: 77%) and advanced adenomas (sensitivity: 58%, specificity: 77%). However, despite its effectiveness, FIT uptake was low: only 7.25% of individuals underwent testing within six months of their baseline clinical exam. This limited adoption limits the potential impact of FIT on early detection and intervention in CRC.

In contrast, CBC tests are routinely performed in clinical practice, offering greater coverage and a more scalable and accessible approach to early detection. In the embodiments of the present invention, the CBC-based model demonstrated 64% sensitivity and 81% specificity for detecting CRC, and 32% sensitivity with the same specificity for advanced adenomas. This suggests that screening 20% of the population aged 45 to 75 could potentially detect 64% of CRC cases and 32% of advanced adenomas.

Although FIT remains more sensitive for detecting both CRC and advanced adenomas, the widespread availability of CBC tests allows the CBC-based model to function as a pre-screening tool. This approach can help identify high-risk individuals who would benefit from additional testing such as FIT or colonoscopy, optimizing resource allocation and improving overall screening adherence.

In conclusion, the present invention demonstrates the potential use of routinely collected CBC data to identify individuals at higher risk for CRC. The CBC-based model can be used as a pre-screening tool to identify high-risk individuals for additional testing with FIT or colonoscopy. This approach can improve resource allocation and increase adherence to screening recommendations.

What is claimed is:

1. A method for training an artificial intelligence model to predict the chances of colorectal carcinoma or advanced colorectal adenoma occurring in an individual, comprising at least the steps of:
receiving a first set of features, wherein the first set of features relates to individuals who have not been diagnosed with any type of colorectal carcinoma or advanced colorectal adenoma and comprises, as features for each individual, at least one feature obtained from the red series of a blood count and/or at least one feature obtained from the white series of a blood count;
receiving a second set of features, wherein the second set of features relates to individuals diagnosed with any type of colorectal carcinoma or advanced colorectal adenoma and comprises, as features for each individual, at least one feature obtained from the red series of a blood count and/or at least one feature obtained from the white series of a blood count;
training the artificial intelligence model based on the first and second sets of features.

2. The method of claim 1, wherein the first and second sets of features additionally comprise age as a feature for each individual.

3. The method of claim 1, wherein the first and second sets of features additionally comprise at least one feature obtained from the platelet series of a blood count.

4. The method of claim 1, wherein the model is trained using a supervised or unsupervised learning algorithm, or comprises a combination of models trained using a supervised and/or unsupervised learning algorithm.

5. A system for training an artificial intelligence model to predict the chances of colorectal carcinoma or advanced colorectal adenoma occurring in an individual, comprising at least one processor, wherein the processor is configured to carry out the method as defined in claim 1.

6. A computer-readable medium comprising instructions that, when executed by at least one processor, cause the processor to carry out the method as defined in claim 1.

7. A method for predicting the chances of colorectal carcinoma or advanced colorectal adenoma occurring in an individual, comprising the steps of:
receiving, as features for the individual, at least one feature obtained from the red series of a blood count and/or at least one feature obtained from the white series of a blood count;
calculating, using a model trained according to the method as defined in claim 1 and based on the received features, the chances of colorectal carcinoma or advanced colorectal adenoma occurring in the individual.

8. The method of claim 7, wherein the first and second sets of features additionally comprise age as a feature for the individual.

9. The method of claim 7, wherein the first and second sets of features additionally comprise at least one feature obtained from the platelet series of a blood count.

10. A system for predicting the chances of colorectal carcinoma or advanced colorectal adenoma occurring in an individual, comprising at least one processor, wherein the processor is configured to carry out the method as defined in claim 7.

11. A computer-readable medium comprising instructions that, when executed by at least one processor, cause the processor to carry out the method as defined in claim 7.
